(19) United States
(12) Patent Application Publication
(10) Pub. No.: US 2025/0339049 A1
(43) Pub. Date: Nov. 6, 2025

(54) A SYSTEM AND METHOD FOR DETERMINING AN EMOTIONAL STATE OF A USER BASED ON ONE OR MORE PHYSICAL AND/OR PHYSIOLOGICAL PARAMETERS

(71) Applicant: SIGNIFY HOLDING B.V., EINDHOVEN (NL)

(72) Inventors: JIN YU, LEXINGTON, MA (US); MUHAMMAD MOHSIN SIRAJ, EINDHOVEN (NL); PETER DEIXLER, ARLINGTON, MA (US)

(21) Appl. No.: 18/869,973

(22) PCT Filed: May 22, 2023
(86) PCT No.: PCT/EP2023/063611

§ 371 Date: Nov. 27, 2024

Related U.S. Application Data
(60) Provisional application No. 63/347,455, filed on May 31, 2022.

(30) Foreign Application Priority Data
Jun. 14, 2022 (EP) 22178852.4

(51) Int. Cl. A61B 5/0507; A61B 5/11; A61B 5/16

(52) U.S. Cl. CPC A61B 5/0507; A61B 5/1113; A61B 5/165

(57) ABSTRACT

A method and system for determining an emotional state of a user based on a radiofrequency, RF, based sensing system are disclosed. The system comprises a presence detection unit configured to receive presence data and to determine a presence of the user and a visitor in an environment. The system further comprises a controller configured to receive a first and second input indicative of the presence of the user and the visitor respectively. The controller is further configured to, when the user is present, set the RF-based sensing system to a first operating mode, and when the user and the visitor are present, switch the RF-based sensing system to a second operating mode, wherein, when set to the second operating mode, the RF-based sensing system operates according to a second set of RF-sensing parameters that enable to sense one or more physical and/or physiological parameters of the user. The controller is further configured to obtain the RF signals when the RF-based sensing system is set to the second operating mode, determine the one or more physical and/or physiological parameters of the user based on the RF signals, and determine the emotional state of the user is an elevated emotional state based on the one or more physical and/or physiological parameters, wherein an elevated emotional state is an emotional state that deviates from a baseline emotional state of the user.

### FIELD OF THE INVENTION

The invention relates to a method for determining an emotional state of a user based on a radiofrequency-based sensing system. The invention further relates to a system for determining an emotional state of a user. The invention further relates to a computer program for determining an emotional state of a user based on a radiofrequency-based sensing system.

### BACKGROUND

Elder mistreatment includes intentional or neglectful acts by a caregiver or trusted person that harm a vulnerable older person. Elder mistreatment may manifest in a combination of forms, including emotional abuse, physical abuse, sexual abuse, financial exploitation, and neglect and abandonment. Furthermore, elder mistreatment is associated with multiple serious consequences, including increased rates of physical injuries, depression, emotional distress, functional decline, emergency department use, hospital admissions, and morbidity and mortality. The public health impacts of elderly abuse may be far-reaching due to the numerous and varied physical and psychosocial consequences of being exposed to these phenomena.

Child maltreatment and abuse is well established as an important societal concern with significant ramifications for the affected children, their families, and society at large. Incidents of child maltreatment and abuse often remain under-reported as children often are reluctant to tell about abuse.

### SUMMARY OF THE INVENTION

The inventors have realized that when a user experiences abuse by a visitor, for instance a caregiver or trusted person, he/she may show signs of physiological arousal (e.g., elevated levels of one or more physical and/or physiological parameters such as elevated heart rate, respiratory rate, increased body motion, pupil dilation) in response to the abuse-related stimuli. However, state-of-the-art solutions for monitoring such physical and/or physiological parameters, e.g., photoplethysmography, electrocardiography, plethysmography, etc., are obtrusive, while remote camera-based solutions may pose serious privacy and legal considerations. Detecting such physiological arousal can be performed unobtrusively using radiofrequency (RF) based sensing. However, when a visitor is present, adjusting RF sensing (configuration) parameters may be required to optimally monitor the physical and/or physiological parameters of the user.

Generally, RF based sensing may be performed with respect to a plurality of different applications and different sensing goals, wherein each of these applications and sensing goals requires a different configuration of the radiofrequency sensing. For example, if the sensing goal refers to a simple presence detection task for controlling the lights in a room, the accuracy of the detection is not necessarily very high and the algorithm used for the radiofrequency sensing can, for instance, be based on an RSSI value of the different radiofrequency signals. However, more advanced applications, for instance, respiratory monitoring of the user in a specific area of an environment may often require a much higher accuracy together with a respective higher spatial resolution. In such cases, the RF sensing can be performed based on an algorithm using a CSSI of the RF signals wherein additionally specific signal paths are weighted stronger than other signal paths. Generally, the differences in the RF sensing parameters and the specific configurations of the RF sensing to a specific application and sensing goal can be regarded as different RF sensing modes. Accordingly, as becomes apparent from the examples, a different operating mode of the RF based sensing system may be used to optimally monitor the physical and/or physiological parameters of the user. For example, more resources may be required. For instance, additional power and bandwidth may be required to better monitor the activities of the user and visitor, certain (configuration) parameters such as carrier frequency may need to be adjusted to avoid interference, etc. The inventors have realized that by adjusting the RF sensing parameters when the visitor is present, the performance of the monitoring of the physical and/or physiological parameters is more accurate. It is therefore an object to provide a more accurate system to determine an emotional state of a user based on the presence of a visitor.

According to a first aspect, the object is achieved by a system for determining an emotional state of a user, the system comprising a radiofrequency, RF, based sensing system comprising one or more nodes arranged for transmitting and/or receiving RF signals for RF-based sensing, a presence detection unit configured to receive presence data and determine, based on the received presence data, a presence of the user in an environment and further configured to determine, based on the received presence data, a presence of a visitor in the environment and a controller configured to:
receive a first input indicative of the presence of the user in the environment and further receive a second input indicative of the presence of the visitor in the environment;
when the user is present, set the RF-based sensing system to a first operating mode, wherein, when set to the first operating mode, the RF-based sensing system operates according to a first set of RF-sensing parameters;
when the user and the visitor are present, switch the RF-based sensing system to a second operating mode, wherein, when set to the second operating mode, the RF-based sensing system operates according to a second set of RF-sensing parameters, different from the first set of RF-sensing parameters, wherein the second set of RF-sensing parameters enable to sense one or more physical and/or physiological parameters of the user;
obtain the RF signals when the RF-based sensing system is set to the second operating mode;
determine the one or more physical and/or physiological parameters of the user based on the RF signals; and
determine if the emotional state of the user is an elevated emotional state based on the one or more physical and/or physiological parameters, wherein an elevated emotional state is an emotional state that deviates from a baseline emotional state of the user.

When people experience abuse by a visitor, for instance a caregiver or trusted person, certain physical and/or physiological parameters may change in response to the abuse-related stimuli. For example, when elderly people, babies/toddlers experience abuse by trusted persons/caregivers, pets experience abuse by trusted trainers/sitters, employees experience any form of emotional or physical abuse by employers, etc., certain physical and/or physiological parameters such as heart rate, respiratory rate, body motion, etc., may change in response to the undergoing situation. For example, a baby may show elevated levels of distress manifested as elevated heart rate or breathing rate when experiencing physical abuse by a trusted caregiver. In a further example, a pet undergoing physical abuse by a trainer may show similar manifestation of physical and/or physiological parameters. Thus, such changes in the physical and/or physiological parameters may be indicative of an elevated emotional state of the user. In the context of the present disclosure, the term ‘elevated emotional state’ may refer to an emotional state that deviates from a baseline emotional state of the user. The emotional state may be defined by one or more values indicative of a level of arousal and/or valence, and the baseline emotional state may be a baseline value indicative of a baseline level of arousal and/or valence. Examples of elevated emotional states may include but are not limited to anger, stress, excitement, depression, happiness, sadness, etc. For example, the heart rate of the user may increase significantly when the user is angry or scared, but the heart rate may decrease significantly in an emotional state of disgust. The heart rate variability (HRV) may be significantly larger in an amused emotional state than in a fear, neutral, or angry emotional state. Additionally, or alternatively, the respiratory rate of the user may change based on emotional changes. For example, an increase in the respiratory rate may be noted when the user (adult, baby, pet, etc.,) experiences emotional and/or physiological stress or when(s) he feels excited, and the levels of user anxiety may affect the respiratory rate, especially the expiratory time. Wirelessly connected nodes arranged for transmitting and/or receiving radiofrequency, RF, signals may be used for RF-based sensing, such as motion and presence detection, but also for vital sign monitoring, for example, heart rate and respiratory rate monitoring.

The RF based sensing system can be configured to operate at different operating modes, for example, by using different sets of RF-sensing (configuration) parameters and/or algorithms at different times. For example, a first set of RF-sensing (configuration) parameters may be used for detecting basic motion when no visitor is present, a different set of RF sensing parameters may be used for detecting the user's gestures and falls, a second set of RF sensing parameters may be used for determining respiratory rate if a visitor is present, etc. RF-based monitoring of physiological parameters, e.g., heart rate, respiratory rate, etc., typically requires more resources, e.g., increased data rate, an increased number of nodes, more computationally demanding time-frequency signal analysis, etc., compared to simple presence detection or people counting tasks. For example, temporal drifts in RSSI/CSI cab be used for respiratory rate detection as breathing motion events take a long time to be determined, e.g. ˜20 human breaths per minute, whereas such high resolution temporal processing may be unnecessary when trying to determine occupancy, hand gestures or falls to the ground, as those events take at most 1 or 2 seconds. Additionally, and/or alternatively, certain parameters of the RF-based sensing system may be advantageously adjusted to avoid interference by external sources, e.g., the visitor.

The controller is configured to set, when only the user is present, the RF-based sensing system to a first operating mode, wherein, when set to the first operating mode, the RF-based sensing system operates according to a first set of RF-sensing parameters. For example, when only the user is present the RF-based sensing system may be configured to detect the presence of the user in an environment, perform posture determination of the user, count the number of people in the environment, etc. Upon the detection of a visitor, e.g., a caregiver, a trusted person, etc., the controller may switch the RF-based sensing system to a second operating mode, wherein, when set to the second operating mode, the RF-based sensing system operates according to a second set of RF-sensing (configuration) parameters, different from the first set of RF-sensing parameters, wherein the second set of RF-sensing parameters are for sensing one or more physical and/or physiological parameters (e.g., heart rate, respiratory rate, pupil size, etc.) of the user that are indicative or reflective of an emotional state of the user. By switching from a first to a second operating mode, wherein the RF-sensing parameters of the second operating mode are selected for monitoring the one or more physiological parameters of the user when the visitor is present, a more efficient and more accurate way to monitor the one or more physiological parameters of the user is provided.

The controller may be configured to adjust/select the second set of RF-sensing parameters, such that the performance of the RF-based sensing system is optimized for sensing the one or more physical and/or physiological parameters of the user. The one or more physical and/or physiological parameters may comprise one or more of: heart rate, respiratory rate and body movement. Since the second set of RF-sensing parameters are adjusted for the task of monitoring the physical and/or physiological parameters of interest, the performance and reliability of the RF-based sensing system is optimized for the application of interest.

The controller may be configured to receive input indicative of a position and/or orientation of the one or more nodes with respect to the user and/or the visitor and adjust/select the second set of RF-sensing parameters such that interference from the presence of the visitor is avoided. The controller can for example be configured for selecting a ceiling mounted node compared to a wall mounted node, as the wall mounted node's field of view may be blocked by the visitor. Since the controller may be configured for adjusting/selecting the second set of RF-sensing parameters such that the interference from the presence of the visitor for sensing the one or more physical and/or physiological parameters of the user is avoided, the performance of the system for sensing the one or more physical and/or physiological parameters of the user is improved.

The presence detection unit may be configured to determine the presence of the user and/or the presence of the visitor in the environment based on the RF signals transmitted and/or received by the RF-based sensing system when the RF-based sensing system is set to the first operating mode. For example, the controller may be configured to select/adjust the first set of RF-sensing parameters for presence detection or people counting. Since the RF-based sensing system can be used for detecting the presence of the user and/or the visitor, and also for monitoring the one or more physical and/or physiological parameters of the user, the complexity of existing systems for determining an emotional state of a user can be reduced.

The presence detection unit may determine the presence of the user and/or the visitor based on input from a user interface for manually entering the presence of the user or the start of a visitor's visit to the user. For instance, the user, the visitor, or a person working in the surrounding of the user (e.g., hospital staff) may simply press a button when the user enters the environment and/or when a visit starts. Additionally, and/or alternatively, the presence detection unit may determine the presence of the user and/or the visitor based on presence data from one or more occupancy sensor devices located in the environment. This allows to automatically recognize the presence of the user and/or the visitor in the environment.

The controller may be configured to determine if the emotional state of the user is an elevated emotional state by applying a machine-learning model to the one or more physical and/or physiological parameters. The machine-learning model can be trained to determine an elevated emotional state using the one or more physical and/or physiological parameters as inputs. For example, a neural-network, probabilistic, regression model, etc., may be trained using as input, the one or more physical and/or physiological parameters and as output, labeled instances of elevated emotional state, i.e., labeled as stressed, angry, happy, etc.

Certain user activities, such as toileting, outgoing, sleeping activities, etc., may be related/indicative of an elevated emotional state of the user. The controller may be further configured to receive activity data indicative of user activities over a prolonged observation period, extract features indicative of user activities over the observation period based on the received activity data and determine if the emotional state of the user is an elevated emotional state by applying a machine-learning model, such as a Support Vector Machine, a Neural Network, a Light Gradient Boosting Machine (LGBM), etc., on the extracted features. This improves the accuracy of the system on determining if the emotional state of the user is an elevated emotional state.

Long term abuse or abuse-related trauma may be reflected in pattern changes in certain activities of the user (e.g., changes in toileting, outgoing and sleeping). The controller may be configured to receive activity data indicative of user activities over an observation period, extract features indicative of user activities over the observation period based on the received activity data and determine the occurrence of an abuse incident by applying a machine-learning model on the extracted features. The machine-learning model can be trained to detect a probability of an abuse incident based on the extracted features over the observation period. By detecting behavior change over time, early symptoms, or trauma effect due to abuse may be detected, enabling to provide timely interventions to the user and stop the abuse.

The controller may be configured to output a signal indicative of the emotional state of the user to a target device. For example, if the emotional state of the user is an elevated emotional state, the user, a hospital, a caregiver facility, etc., may be notified by a message at a device, such as a personal smart phone or other digital interface (e.g., web user interface). Additionally, and/or alternatively, the controller may be configured to output aggregations of the emotional state of the user, for example, on a dashboard with key emotional state indicators and/or the one or more physical and physiological parameters in a numeric or graphical form. Such dashboard could be presented on a digital user interface (e.g., web user interface).

The controller may be configured to receive data indicative of a visitor identification of the visitor and associate the (elevated) emotional state of the user with the visitor identification. The data may comprise video or voice recordings that allow the identification of the visitor based on for example face and/or voice recognition, recognition of clothing of people working in a care facility/hospital, etc. Additionally, or alternatively, the data may comprise body mass estimation, respiratory rate and/or heart-rate recordings of the visitor that allow the identification of the visitor based on the visitor's body mass, respiratory rate and/or heart-rate pattern. Associating the emotional state of the user with the visitor identification allows to identify candidate/possible abusers. For example, in an assisted living facility/hospital, a specific caregiver that may intentionally or unintentionally mistreat elders, can be identified by correlating the users showing signs of abuse with a specific visitor/care giver's identification across many different rooms in an assisted living facility/hospital.

The set of RF-sensing parameters may comprise one or more of: a reception sensitivity, an orientation of the one or more of the nodes, an amount of nodes at different relative locations, a connectivity of the one or more of the nodes to one or more other nodes, a frequency channel or frequency channels, a transmission power, a transmit beam shaping, a receive beam shaping and bandwidth.

The orientation of the one or more of the nodes may include an orientation of the one or more of the nodes with respect to a specific volume, to one or more other nodes, to the user, to the visitor, or a combination thereof. For example, an RF-sensing node may adjust its detection area using beamforming. The coverage of the environment may be increased by increasing the power of the RF-sensing node. The connectivity of the one or more of the nodes to one or more other nodes may include a data transfer rate between the nodes, such as a data transfer rate required for heart-rate monitoring in real time. For example, if the data transfer rate between the nodes is below the one required for heart-rate detection in real time, RF-sensing parameters may be adjusted in order to increase the data transfer rate. The connectivity of the one or more of the nodes to one or more other nodes may also for example include a number of retries for transmitting data. For example, the nodes may have a worse connectivity if a higher number of retries is required for transmitting data. The amount of nodes at different relative locations may refer to an amount of nodes at different relative locations in the environment relative to the user or an amount of nodes at different relative locations of a group of nodes in the environment. A group of nodes may for example include a group of or all nodes arranged in a room, on a floor, in a house, or the like. For example, a group of nodes may include a ceiling mounted node and four table nodes. If two of the four table nodes are located in an area of the room that the line-of-sight to the user is blocked, for example by the presence of the visitor, then only the ceiling mounted node and the two table nodes are selected for RF-based sensing in the second operating mode. This improves the performance of sensing the one or more physical and/or physiological parameters of the user.

In many cases, the user may continue to show elevated levels of one or more physical and/or physiological parameters for a time period after the end of a visit from the visitor. The controller may further be configured to receive data indicative of the end of the visitor's visit to the user and switch the RF-based sensing system to the first operating mode after a time period after the end of the visit. This time period may be fixed, or dynamic based on the determined emotional state of the user during the visit. Monitoring the one or more physical and/or physiological parameters of the user for a time period after the end of the visit allows to estimate the recovery time of the user after the end of the visit, e.g., the time elapsed from an elevated emotional state to a baseline emotional state of the user after the visit.

According to a second aspect, the object is achieved by a method for determining an emotional state of a user based on an RF-based sensing system, the RF-based sensing system comprising one or more nodes arranged for transmitting and/or receiving RF signals for RF-based sensing, the method comprising the steps of:
receiving a first input indicative of a presence of the user in an environment;
receiving a second input indicative of a presence of a visitor in the environment;
when the user is present, setting the RF-based sensing system to a first operating mode, wherein, when set to the first operating mode, the RF-based sensing system operates according to a first set of RF-sensing parameters;
when the user and the visitor are present, switching the RF-based sensing system to a second operating mode, wherein, when set to the second operating mode, the RF-based sensing system operates according to a second set of RF-sensing parameters, different from the first set of RF-sensing parameters, wherein the second set of RF-sensing parameters are adjusted for sensing one or more physical and/or physiological parameters of the user;
obtaining the RF signals when the RF-based sensing system is set to the second operating mode;
determining the one or more physical and/or physiological parameters of the user based on the RF signals;
determining if the emotional state of the user is an elevated emotional state based on the one or more physical and/or physiological parameters, wherein an elevated emotional state is an emotional state that deviates from a baseline emotional state of the user.

According to a third aspect, the object is achieved by a computer program that is configured to perform a method for determining an emotional state of a user based on an RF-based sensing system.

It should be understood that the system, method and computer program product may have similar and/or identical embodiments and advantages as the above-mentioned lighting devices.

### BRIEF DESCRIPTION OF THE DRAWINGS

The above, as well as additional objects, features and advantages of the disclosed systems, devices and methods will be better understood through the following illustrative and non-limiting detailed description of embodiments of devices and methods, with reference to the appended drawings, in which:

FIG. 1 shows schematically a block diagram of a system for determining an emotional state of a user;

FIG. 2 shows schematically an example of a system for determining an emotional state of a user;

FIG. 3 shows schematically a flow diagram of a method of determining an emotional state of a user.

All the figures are schematic, not necessarily to scale, and generally only show parts which are necessary in order to elucidate the invention, wherein other parts may be omitted or merely suggested.

### DETAILED DESCRIPTION

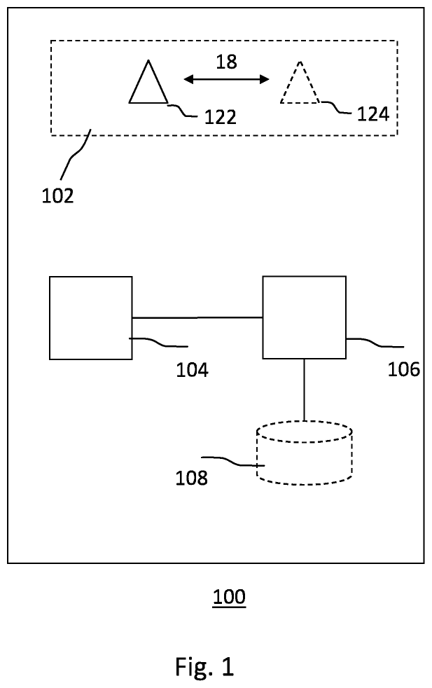

FIG. 1 shows an example of a system 100 for determining an emotional state of a user. The system 100 comprises a radiofrequency, RF, based sensing system 102 comprising one or more nodes 122, 124 arranged for transmitting and/or receiving RF signals 18 for RF-based sensing. The one or more nodes 122, 124 may for example be in the form of a Wi-Fi transceiver node 122 arranged for transmitting RF signals 18 to the receiver node 124, in the form of a radar sensor 122 arranged for transmitting and receiving RF signals 18, in the form of a group of radar sensors 122, 124 arranged for transmitting and receiving RF signals 18, etc.

The system 100 further comprises at least one data processor or controller 106. The system 100 may further comprise at least one data repository or storage or memory 108 for storing computer program code instructions.

The system 100 further comprises a presence detection unit 104 configured to receive presence data, such as RF signals, input signals from a user interface, data from one or more occupancy sensors, e.g., camera, audio-recording device, PIR sensors, CO2 sensors, vibration sensors, RFID tags, etc., and to determine, based on the received presence data, the presence of the user in an environment. The presence detection unit 104 is further configured to determine, based on the received presence data, the presence of a visitor to the user in the environment. The presence detection unit 104 can be integrated in the controller 106 or may be a separate device (e.g., a wearable device in the form of a button or touch screen so that the user can enter the start of a visit, a sensing arrangement, e.g., a camera, configured for sensing the presence of people in the environment, a presence sensor, etc.).

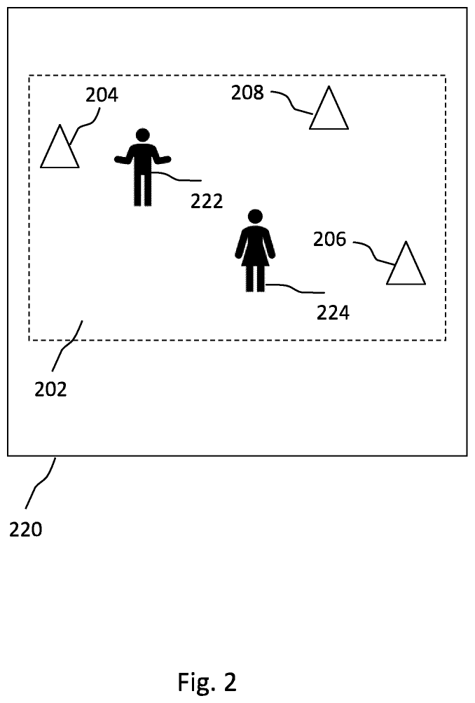

FIG. 2 shows schematically an example of a system for determining an emotional state of a user. The controller 106 is configured to receive a first input indicative of the presence of a user 222 in an environment 220. The environment 220 can be a room which may be limited by walls, floor and ceiling, an open-space arrangement, a corridor, etc. When the user 222 is present in the environment 220, the controller 106 is configured set the RF-based sensing system to a first operating mode. When set to the first operating mode, the RF-based sensing system 202 comprising the nodes 204, 208, 206 operates according to a first set of RF-sensing (configuration) parameters. For example, the set of parameters of the RF based sensing system may be selected to perform the task of presence detection of the user 222. Additionally, or alternatively, the set of parameters of the RF based sensing system may be selected to perform people counting in the environment 220, determination of the posture of the user 222, etc. The controller 106 is further configured to receive a second input indicative of the presence of a visitor 224 in the environment 220. Upon the detection of the visitor 224 in the environment 220, the controller 106 is configured to switch the RF-based sensing system 202 to a second operating mode. When set to the second operating mode, the RF-based sensing system 202 operates according to a second set of RF-sensing (configuration) parameters, different from the first set of RF-sensing parameters, wherein the second set of RF-sensing parameters enable to sense one or more physical and/or physiological parameters of the user 222. The controller 106 is configured to obtain the RF signals and determine the one or more physical and/or physiological parameters of the user, e.g., heart rate, respiratory rate, body mass index, etc., based on the RF signals.

For example, a doppler radar sensing system 202, comprising a group of nodes 204, 206, 208, may be used for respiratory monitoring by transmitting signals 18, and by receiving reflected signals 18 with a Doppler shift caused by a periodic motion of the user's chest. The respiratory rate of the user 222 can be extracted from the Doppler shift. Respiratory rate monitoring may require that the radar sensing system 202 uses higher transmission power relative to the transmission power of the nodes when the system operates in the first operating mode and/or specific frequency bandwidth (as the respiration rate falls within a certain range) for accurate monitoring. Thus, the RF-sensing parameters of the RF sensing system 202 may be appropriately switched from the first set of RF-sensing parameters to the second set of RF-sensing parameters configured for the task of respiratory rate monitoring.

Alternatively or additionally, the RF sensing system 202 may comprise 802.15.4 compliant or Wi-Fi compliant wireless sensor nodes 204, 206, 208 and the received signal strength (RSS) signals 18 transmitted between the wireless sensor nodes 204, 206, 208 may be used for respiratory rate or heart rate monitoring. A higher number of RF sensor nodes compared to the first operating mode may be required (thus more resources) for accurate monitoring. In another example, only RF sensor nodes in close proximity to the chest of the user 222 may be used for accurate monitoring. Wireless sensor nodes are typically vulnerable to interference from other devices; thus, the RF based sensing system may switch to a different operating frequency compared to the operating frequency in the first operating mode to avoid interference from other devices.

The controller 106 is configured to determine if the emotional state of the user 222 is an elevated emotional state based on the one or more physical and/or physiological parameters. For example, if the user 222 experiences abuse by the visitor 224 (the visitor can be a health-care provider, e.g., a nurse, a doctor, etc., a trusted person, etc.), certain physical and/or physiological parameters such as heart rate, respiratory rate, body motion, etc., may change in response to the abuse-related stimuli. Thus, such physical and/or physiological parameters may be indicative of/correlated to an elevated emotional state of the user 222. For example, the user 222 might experience an elevated heart rate, compared to the baseline heart-rate measurements of the user. Thus, the controller may determine that the emotional state of the user is a stressed emotional state in response to the presence of the visitor 224.

The presence of the visitor 224 in the environment 220 may hinder the accuracy of the RF based sensing system 202 for monitoring the one or more physical and/or physiological parameters of the user 222. For example, the visitor 224 may be positioned in the line-of-sight between sensor node 206 and the user 222, which may degrade the accuracy of the RF based system 202 for monitoring the one or more physical and/or physiological parameters of the user 222. The controller 106 may be configured to receive input indicative of a position and/or orientation of the one or more nodes with respect to the visitor 224 and the user 222 and select the second set of RF-sensing parameters for avoiding interference due to the presence of the visitor 224 to sense the one or more physical and/or physiological parameters of the user 222. For example, the controller 106 may be configured for selecting only nodes 204 and 208, as the RF signal transmission of node 206 may be blocked by the visitor 224.

The position and/or orientation of visitor 224 with respect to the user 222 may be used by the controller 106 for selecting the second set of RF-sensing parameters for avoiding interference. In the illustrative example of FIG. 2, the user 222 may be oriented towards node 208 and the visitor 224 towards node 206, respectively. However, the difference of the distances between the chests of the user 222 and the visitor 224 to node 208 may be such that it may be difficult to identify the user's respiratory signal using node 208. Therefore, the controller 106 may select node 204, whose orientation angle is the second closest to the user 222, for monitoring the one or more physical and/or physiological parameters of the user 222.

The position and/or orientation of nodes can be manually or automatically determined. The position of a node can for example be manually inserted by the user, e.g., via a user interface, such as a user input device with a touch screen or display and keyboard. The position and/or orientation can also be determined automatically, e.g., based on tracking the position and environment of the node, e.g., via a camera, or in any other manner known to the skilled person. Alternatively, or additionally, the position and/or orientation can be a node parameter stored for each node on the node, e.g., during production of the node, arrangement of the node, or both. Alternatively, or additionally, the nodes can be configured for determining their position and/or orientation. The nodes may be configured for providing/transmitting their position to the controller 106. Techniques for determining the locations of nodes and/or people are known in the art and will therefore not be discussed in further detail.

The RF based sensing system 202 may be configured to operate in a first operating mode, wherein when set to the first operating mode, the RF-sensing parameters are adjusted for performing presence detection. For example, presence detection of the user 222 and/or the visitor 224. The RF signals 18 transmitted and received by the nodes comprising the RF based sensing system 202 may be received and analyzed by the presence detection unit 104 to determine the presence of the user 222 and/or the presence of the visitor 224 in the environment 220. For example, by analyzing disturbances of the RF signals 18 caused by the presence of the user 220 and/or the visitor 224.

Additionally, or alternatively, the presence detection unit 104 may be configured to receive an input signal from a user interface. The user interface may comprise a simple button, dedicated software, e.g., a visitor registration software, etc. For instance, the user, the visitor, or a person working in the surrounding of the user (e.g., a nurse, doctor, etc.) may press a button or manually enter in the dedicated software when the user 222 and/or the visitor 224 is present in the environment 202.

Additionally, or alternatively, the presence detection unit 104 may receive data from one or more occupancy sensors (a cameras, microphones, CO2 sensors, vibration sensors, a PIR sensing system, radar sensors, etc.) distributed in the environment 202 and determine the presence of the user 222 and/or the presence of the visitor 224 based on the received presence data. For example, a people counting algorithm may be implemented by the controller 106 to analyze received presence data from a PIR sensing system to count the number of people in the environment 202 and thus recognize the presence of the user 222 and/or the visitor 224. The presence detection unit 104 may differentiate, based on the received data, the presence of the user 222 from the presence of the visitor 224. For example, a face recognition algorithm may be implemented by the presence detection unit 104 to analyze received video data from a camera to discriminate the user 222 and/or the visitor 224 (differentiate between the user 222 from the visitor 224). In another example, a voice recognition algorithm may be implemented by the presence detection unit 104 to analyze received audio data for the same purpose. In yet another example, the presence detection unit 104 may determine and distinguish the presence of the user 222 and/or the visitor 224 from data received from one or more occupancy sensors detecting signals transmitted from personal devices, e.g., phone, smart watch or BLE-beacon equipped badges associated with the user 222 and/or the visitor 224. In a further example, a breathing rate and/or heart rate signature associated with the user 222 and/or the visitor 224 may be used to differentiate between the user 222 and the visitor 224. Those methods are only mere examples. Several techniques for determining presence and differentiating between occupants in a space based on occupancy sensor data are known in the state of art.

The controller 106 may be configured to determine if the emotional state of the user 222 is an elevated emotional state (e.g., sad, stressed, angry, happy, etc.) by applying a machine-learning model to the one or more physical and/or physiological parameters. For example, the determined one or more physical and/or physiological parameters of the user 222 may be used as input to the machine learning model to determine whether the current emotional state of the user 222 is an elevated emotional state. The trained machine learning model may make such a determination because the machine learning model may have already been trained with inputs that may include instances (time series data) of the one or more physical and/or physiological parameters of the user 222 or instances (time series data) of the one or more physical and/or physiological parameters from a group of similar users (e.g., users that share a similar cultural background, age group, medical/psychiatric condition, etc.), and output corresponding labeled instances of elevated emotional states of the user or a group of similar users.

Changes in the pattern of certain activities, such as toileting, outgoing, sleeping activities, etc., may be indicative of an elevated emotional state of the user. The controller 106 may be configured to receive activity data indicative of user activities over an observation period. For example, the RF-based sensing system 202, motion sensors, such as PIR, single pixel thermopile sensors, etc., may be used to detect minor, major, and medium motions of the user 222. The controller 106 may apply a feature learning algorithm on the time series of received activity data over an observation period (e.g., a week, a month, etc.) to extract features related or indicative of activities of the user. The features may include, but are not limited to, the total number of performing an activity, the frequency of the activity, the rolling average number of the activity per day over an observation period, etc. The controller 106 may be configured to determine if the emotional state of the user 222 is an elevated emotional state (e.g., sad, stressed, angry, happy, etc.) by applying a machine-learning model to the one or more physical and/or physiological parameters and the extracted features indicative of user activities over an observation period. For example, the extracted features from the received activity data and the determined one or more physical and/or physiological parameters of the user may be used as input to the machine learning model to determine whether the current emotional state of the user is an elevated emotional state. The trained machine learning model may make such a determination because the machine learning model may have already been trained with inputs that may include both instances of the one or more physical and/or physiological parameters and series of instances of activity data over time and output corresponding labeled instances of elevated emotional state of the user or the general population.

Long term abuse or abuse-related trauma may be reflected in pattern changes in certain activities of the user (e.g., changes in toileting, outgoing and sleeping). For example, if the user does not drink sufficient water during the day, this will cause dehydration and result in an increase in the number of toiletings. The controller 106 may be configured to receive activity data over an observation period, extract features indicative of user activities over the observation period based on the received activity data and determine the occurrence of an abuse incident by applying a machine-learning model on the extracted features. The machine learning model may be trained to determine the probability of abuse or abuse-related trauma based on labeled extracted features during abusive incidents or situations.

The controller 106 may be configured to output a signal indicative of the emotional state of the user 222 to a target device. The output signal may have different forms. For example, the output signal may be a message and/or notification to a device such as a personal smart phone or other digital interface if the emotional state of the user is determined to be an elevated emotional state. In another example, a notification may be sent to a digital interface located in a caregiver facility when a user 222 is experiencing stress. The output signal may be in the form of a visual notification, e.g., a luminaire blinking, an audible signal like a special tone and/or a sensible warning like a vibration, for example in the case that the emotional state of the user is an elevated emotional state. Additionally, or alternatively, the user may be notified by a message in a personal device, e.g., a smartphone, that(s) he is angry. Additionally, or alternatively, the controller 106 may be configured to output aggregations of the emotional state of the user, for example, on a dashboard with key emotional state indicators and/or the one or more physical and physiological parameters in a numeric or graphical form. Such a dashboard may be presented on a digital user interface (e.g., web user interface). For example, the controller 106 may output stress, anger, etc., levels of the user 222 over an observation period. In a further example, the controller 106 may output the determined heart rate or breathing rate of the user 222 in a graphical form.

The controller 106 may be configured to receive data indicative of a visitor identification of the visitor 224 and associate the (elevated) emotional state of the user 222 with the visitor identification. For example, the data may comprise an RFID tag to identify the visitor 224, e.g., person working in a hospital, care facility, etc. In another example, the data may comprise video or voice recordings of the environment 220. The controller 106 may for example apply face and/or voice recognition, recognition of clothing of people working in a care facility/hospital, to identify the visitor 224. Additionally, and/or alternatively, the data may comprise the RF signals 18 transmitted and/or received by the RF based sensing system 102. The controller 106 may for example determine certain physical and/or physiological parameters, such as respiratory rate, body mass index, etc., of the visitor 224 that allow the identification of the visitor 224. For example, a visitor 224 may be identified based on his/her body mass and unique respiratory rate and/or heart-rate pattern.

The controller 106 may be configured to select the second set of RF sensing parameters of the RF based sensing system 102 for monitoring the one or more physical and/or physiological parameters of the user. The set of RF sensing parameters that may be adjusted/selected may for example include a reception sensitivity, an orientation of the one or more of the nodes, an amount of nodes at different relative locations, a connectivity of the one or more of the nodes to one or more other nodes, a frequency channel or frequency channels, a transmission power, a transmit beam shaping, a receive beam shaping, and bandwidth.

For example, if breathing rate detection of the user 222 is desired while both the user 222 and the visitor 224 are present in the environment 220, the wireless communication signal may need to be spatially confined to determine the small breathing-related chest movement of the user 222 while keeping the electromagnetic radiation as much as possible away from the visitor 224, for example to avoid interference from hand movements of the user 222 when talking to the visitor 224. In this case, the RF based sensing system 202 may adjust the operating frequency in the second operating mode and operate at a higher channel frequency compared to the channel frequency of the first operating mode, for instance 5 GHz/6 GHz compared to 2.4 GHz. Preferably, the RF sensing based system 201 operates at mm-wave or even THz RF sensing.

The connectivity of the one or more nodes may refer to the data rate that messages are exchanged between the nodes of the RF based sensing system 202. For example, RF wireless communication signals are typically sent with 30 Hz data rate for occupancy & breathing detection, while for heartbeat or fall detection preferably a higher data rate (sampling frequency), e.g. 1000 Hz for fall detection, may be used.

In another example, for the task of respiratory rate detection, a channel bandwidth higher than 80 MHz may be required when operating in the second operating mode. On the contrary, when operating in the first operating mode, for example for the task of occupancy detection, a channel bandwidth of 20 MHz may be sufficient for basic motion sensing.

In another example, the controller 106 may increase the data rate of the RF based sensing system 202 to be able to perform heart rate or respiratory rate monitoring, increase the transmission power of the RF based sensing system 202 to increase coverage, increase the number of retries to increase robustness, use beamforming on the RF based sensing system 202 to adjust the detection area of the nodes, etc. The controller 106 may be configured to adjust/select the second set of RF sensing parameters of the RF based sensing system 102 such that the performance of the RF based sensing system 102 is optimized for the task of monitoring the one or more physical and/or physiological parameters of the user 222.

For example, FIG. 2 shows a situation in which the visitor 224 is blocking the line of sight between node 206 and the user 222. The controller 106 may select only nodes 204 and 206 for monitoring the one or more physical and/or physiological parameters of the user 222, to optimize the performance of the RF based sensing system 202.

It should be understood that the above-mentioned examples of RF sensing parameters for the first or second operating modes are mere examples, and that the skilled person is able to conceive alternatives without departing from the scope of the appended claims.

In many situations, the emotional state of the user 222 may remain elevated for a time period after the end of a visit from the visitor 224. The controller 106 may further be configured to receive data indicative of the end of the visitor's visit to the user 222 and switch the RF-based sensing system to the first operating mode after a time period after the end of the visit. The controller 106 may further be configured to estimate a recovery time, i.e., the time that the user 222 transits from an elevated emotional state to a baseline emotional state, based on the determined one or more physical and/or physiological parameters of the user 222. For example, the controller 106 may continue operate in the second operating mode and monitor the heart rate of the user 222 for a time period after the visit. In this case, the controller 106 may determine the recovery time of the user 222 by summing the time during which the heart rate of the user 222 remained above a predetermined baseline heart rate threshold value. Alternatively, the controller 106 may be configured to obtain the recovery time, for instance from a memory storing the recovery time of the user. The controller 106 may be further configured to switch the RF-based sensing system to the first operating mode after the recovery time.

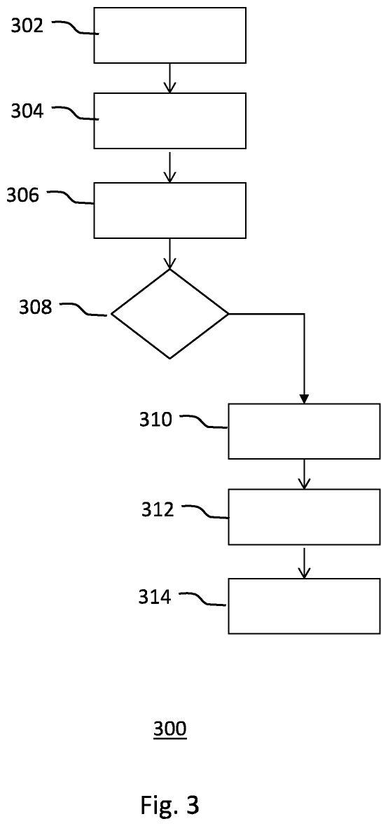

FIG. 3 shows an embodiment of a method 300 for determining an emotional state of a user based on an RF based sensing system, e.g., on the RF based sensing system 102 presented in FIG. 1.

In step 302, a first input indicative of a presence of the user 222 in the environment 220 is received.

In step 304, a second input indicative of a presence of the visitor 224 in the environment 220 is received.

In step 306, the RF-based sensing system is set to a first operating mode.

In a step 308, it is determined whether a visitor is present. If so, the RF-based sensing system is set to a second operating mode.

Steps 310-314 are executed when the RF-based sensing system is set to a second operating mode. Step 310 comprises obtaining the RF signals. Step 312 comprises determining the one or more physical and/or physiological parameters of the user based on the RF signals. Step 314 comprises determining if the emotional state of the user is an elevated emotional state based on the one or more physical and/or physiological parameters.

The method 300 may be executed by computer program code of a computer program product when the computer program product is run on a processing unit of a computing device, such as the controller 106 of the system 100.

It should be noted that the above-mentioned embodiments illustrate rather than limit the invention, and that those skilled in the art will be able to design many alternative embodiments without departing from the scope of the appended claims.

In the claims, any reference signs placed between parentheses shall not be construed as limiting the claim. Use of the verb “comprise” and its conjugations does not exclude the presence of elements or steps other than those stated in a claim. The article “a” or “an” preceding an element does not exclude the presence of a plurality of such elements. The invention may be implemented by means of hardware comprising several distinct elements, and by means of a suitably programmed computer or processing unit. In the device claim enumerating several means, several of these means may be embodied by one and the same item of hardware. The mere fact that certain measures are recited in mutually different dependent claims does not indicate that a combination of these measures cannot be used to advantage.

Aspects of the invention may be implemented in a computer program product, which may be a collection of computer program instructions stored on a computer readable storage device which may be executed by a computer. The instructions of the present invention may be in any interpretable or executable code mechanism, including but not limited to scripts, interpretable programs, dynamic link libraries (DLLs) or Java classes. The instructions can be provided as complete executable programs, partial executable programs, as modifications to existing programs (e.g. updates) or extensions for existing programs (e.g. plugins). Moreover, parts of the processing of the present invention may be distributed over multiple computers or processors or even the ‘cloud’.

Storage media suitable for storing computer program instructions include all forms of nonvolatile memory, including but not limited to EPROM, EEPROM and flash memory devices, magnetic disks such as the internal and external hard disk drives, removable disks and CD-ROM disks. The computer program product may be distributed on such a storage medium, or may be offered for download through HTTP, FTP, email or through a server connected to a network such as the Internet.

1. A system for determining an emotional state of a user, the system comprising:
a radiofrequency, RF, based sensing system comprising one or more nodes arranged for transmitting and/or receiving RF signals for RF-based sensing;
a presence detection unit configured to receive presence data, to determine, based on the received presence data a presence of the user in an environment, and to determine based on the received presence data a presence of a visitor in the environment, and differentiate based on the received data the presence of the user from the presence of the visitor;
a controller configured to:
receive a first input indicative of the presence of the user in the environment and further receive a second input indicative of the presence of the visitor in the environment;
when the user is present, set the RF-based sensing system to a first operating mode, wherein, when set to the first operating mode, the RF-based sensing system operates according to a first set of RF-sensing parameters;
when the user and the visitor are present, switch the RF-based sensing system to a second operating mode, wherein, when set to the second operating mode, the RF-based sensing system operates according to a second set of RF-sensing parameters, different from the first set of RF-sensing parameters, wherein the second set of RF-sensing parameters enable to sense one or more physical and/or physiological parameters of the user;
obtain the RF signals when the RF-based sensing system is set to the second operating mode;
determine the one or more physical and/or physiological parameters of the user based on the RF signals; and
determine if the emotional state of the user is an elevated emotional state based on the one or more physical and/or physiological parameters, wherein an elevated emotional state is an emotional state that deviates from a baseline emotional state of the user.

2. The system of claim 1, wherein the second set of RF-sensing parameters are such that the performance of the RF-based sensing system is optimized for sensing the one or more physical and/or physiological parameters of the user.

3. The system of claim 1, wherein the controller is configured to receive input indicative of a position and/or orientation of the one or more nodes with respect to the visitor and/or the user, and wherein the second set of RF-sensing parameters are for avoiding interference from the presence of the visitor for sensing the one or more physical and/or physiological parameters of the user.

4. The system according to claim 1, wherein:
the presence data are the RF signals transmitted and received by the RF-based sensing system when the RF-based sensing system is set to the first operating mode, wherein the first set of RF-sensing parameters are for presence detection of the user and/or the visitor; and
the presence detection unit is configured to determine the presence of the user and/or the presence of the visitor based on the RF signals.

5. The system according to claim 1, wherein the presence data is one of:
an input signal from a user interface;
data from one or more occupancy sensors located in the environment.

6. The system according to claim 1, wherein the controller is configured to determine if the emotional state of the user is an elevated emotional state by applying a machine-learning model to the one or more physical and/or physiological parameters, wherein the machine-learning model is trained to determine an elevated emotional state based on the one or more physical and/or physiological parameters.

7. The system according to claim 6, wherein the controller is configured to receive activity data indicative of user activities over an observation period and extract features indicative of user activities over the observation period based on the activity data, and wherein the machine-learning model is trained to determine the elevated emotional state based on the one or more physical and/or physiological parameters and the extracted features.

8. The system according to claim 1, wherein the controller is configured to
receive activity data indicative of user activities over an observation period,
extract features from the activity data indicative of user activities over the observation period, and
determine an occurrence of an abuse incident by applying a machine-learning model on the features, wherein the machine learning model is trained to determine a probability of an abuse incident using the features over an observation period as input.

9. The system according to claim 1, the controller further configured to:
output a signal indicative of the emotional state of the user to a target device.

10. The system according to claim 1, wherein the controller is further configured to:
receive data indicative of a visitor identification of the visitor.
associate the emotional state of the user with the visitor identification.

11. The system of claim 1, wherein the set of RF-sensing parameters comprise one or more of: a reception sensitivity, an orientation of the one or more of the nodes, an amount of nodes at different relative locations, a connectivity of the one or more of the nodes to one or more other nodes, a frequency channel or frequency channels, and bandwidth, a transmit beam shaping, a receive beam shaping.

12. The system according to claim 1, wherein the controller is configured to:
receive data indicative of an end of the visitor's visit;
estimate a recovery period of the user based on the one or more physical and/or physiological parameters;
switch the RF-based sensing system to the first operating mode after a time period after the end of the visit.

13. A controller for determining an emotional state of a user, said controller configured to:
receive a first input indicative of a presence of the user in the environment and further receive a second input indicative of a presence of the visitor in the environment, said presence of the user and/or visitor being determined by a presence detection unit based on presence data;
when the user is present, set a radiofrequency, RF, based sensing system to a first operating mode, wherein, when set to the first operating mode, the RF-based sensing system operates according to a first set of RF-sensing parameters, radiofrequency, RF, based sensing system comprising one or more nodes arranged for transmitting and/or receiving RF signals for RF-based sensing;
when the user and the visitor are present, switch the RF-based sensing system to a second operating mode, wherein, when set to the second operating mode, the RF-based sensing system operates according to a second set of RF-sensing parameters, different from the first set of RF-sensing parameters, wherein the second set of RF-sensing parameters enable to sense one or more physical and/or physiological parameters of the user;
obtain the RF signals when the RF-based sensing system is set to the second operating mode;
determine the one or more physical and/or physiological parameters of the user based on the RF signals; and
determine if the emotional state of the user is an elevated emotional state based on the one or more physical and/or physiological parameters, wherein an elevated emotional state is an emotional state that deviates from a baseline emotional state of the user.

14. A method for determining an emotional state of a user based on a radiofrequency, RF, based sensing system, the RF-based sensing system comprising one or more nodes arranged for transmitting and/or receiving RF signals for RF-based sensing, the method comprising the steps of:
receiving a first input indicative of a presence of the user in an environment;
receiving a second input indicative of a presence of a visitor in the environment;
differentiating, based on the first and second inputs, the presence of the user from the presence of the visitor
when the user is present, setting the RF-based sensing system to a first operating mode, wherein, when set to the first operating mode, the RF-based sensing system operates according to a first set of RF-sensing parameters;
when the user and the visitor are present, switching the RF-based sensing system to a second operating mode, wherein, when set to the second operating mode, the RF-based sensing system operates according to a second set of RF-sensing parameters, different from the first set of RF-sensing parameters, wherein the second set of RF-sensing parameters enable to sense one or more physical and/or physiological parameters of the user;
obtaining the RF signals when the RF-based sensing system is set to the second operating mode;
determining the one or more physical and/or physiological parameters of the user based on the RF signals;
determining if the emotional state of the user is an elevated emotional state based on the one or more physical and/or physiological parameters, wherein an elevated emotional state is an emotional state that deviates from a baseline emotional state of the user.

15. A non-transitory computer-readable medium on which are stored a plurality of non-transitory computer-readable instructions that when executed on a processor are configured to perform steps comprising the method of claim 14.
